(19) United States
(12) Patent Application Publication
(10) Pub. No.: US 2014/0330171 A1
(43) Pub. Date: Nov. 6, 2014

(54) METHOD AND DEVICE FOR MONITORING POSTURAL AND MOVEMENT BALANCE FOR FALL PREVENTION

(71) Applicants: National Taiwan University, Taipei City (TW); Industrial Technology Research Institute, Hsinchu (TW)

(72) Inventors: Hsin-Hung Pan, Yilan County (TW); Tung-Wu Lu, New Taipei (TW); Hsuan-Lun Lu, Chiayi City (TW)

(73) Assignees: National Taiwan University, Taipei City (TW); Industrial Technology Research Institute, Hsinchu (TW)

(21) Appl. No.: 14/010,189

(22) Filed: Aug. 26, 2013

(30) Foreign Application Priority Data
May 3, 2013 (TW) 102115872

(51) Int. Cl. A61B 5/11

(52) U.S. Cl. CPC A61B 5/1117; A61B 5/1122
USPC 600/595

(57) ABSTRACT

A method for monitoring postural and movement balance for fall prevent is provided. The method includes the following steps. Multiple sensing signals of a human body are obtained. A center of mass (COM) signal and a center of pressure (COP) signal are modeling according to the sensing signals. A correlation coefficient is calculated according to a mediolateral velocity of the COM signal and the COP signal. A threshold is obtained according to at least one regression model stored in a database. Whether the correlation coefficient is smaller than the threshold is determined. An alert is produced when the correlation coefficient is smaller than the threshold.

### PRIORITY

This application claims the benefit of Taiwan application Serial No. 102115872, filed May 3, 2013, the disclosure of which is incorporated by reference herein in its entirety.

### TECHNICAL FIELD

The disclosure relates in general to an alarm method and device, and more particularly to a method and a device for monitoring postural and movement balance for fall prevention.

### BACKGROUND

The issue of falling of elderly people is paid with much attention with the advent of an aging society. In Taiwan, the occurrence of falling is around 30% for the elderly people above 65 years old, 87% of bone fractures of the elderly people are caused by falling, and the fatality rate of fallers above 85 years old is even as high as 40%. Besides, falling is also one of the main reasons that the elderly people seek emergency medical help, and ranks as a second highest cause of death of the elderly people. Therefore, the impact brought by falling increases not only medical care expenditures but also social care costs.

Falling is often resulted by the loss of balance of the human body. In current clinical practices, detecting static postural balance is confined within professional equipments in hospitals and medical laboratories, and is rather inappropriate for portable uses or even the applications of movement balance monitoring for non-patients (e.g., exercisers).

Therefore, there is a need for a portable device for monitoring postural and movement balance for fall prevention.

### SUMMARY

The disclosure is directed to a method and a device for monitoring postural and movement balance for fall prevention.

According to one embodiment, a method for monitoring postural and movement balance for fall prevention is provided. The method comprises steps of: obtaining a plurality of sensing signals of a human body; modeling the related kinematics of center of mass (COM) signal and center of pressure (COP) signal according to the sensing signals; calculating a correlation coefficient according to a mediolateral velocity of the COM signal and the COP signal; obtaining a threshold according to at least one regression model stored in a database; determining whether the correlation coefficient is smaller than the threshold; and outputting an alarm when the correlation coefficient is smaller than the threshold.

According to another embodiment, a device for monitoring postural and movement balance for fall prevention is provided. The device comprises a sensing module, a calculation processing module, a database and an output module. The sensing module obtains a plurality of sensing signals from a human body. The database stores at least one regression model. The calculation processing module comprises a calculation unit and a determination unit. The calculation unit models related kinematics of COM signal and COP signal according to the sensing signals, and calculates a correlation coefficient according to a mediolateral velocity of the COM signal and the COP signal. The determination unit obtains a threshold according to the regression model, and determines whether the correlation coefficient is smaller than the threshold. The output module outputs an alarm when the correlation coefficient is smaller than the threshold.

### BRIEF DESCRIPTION OF THE DRAWINGS

FIG. 1 shows a schematic diagram of a device for monitoring postural and movement balance for fall prevention.

FIG. 2 shows a detailed block diagram of a sensing module.

FIG. 3 shows a relationship diagram between a vertical acceleration and time.

FIG. 4 shows a schematic diagram of a one-leg standing period when ascending the stairs by simulating a human body with an inverted pendulum model.

FIG. 5 shows a relationship between a vertical acceleration and time.

FIG. 6 shows a relationship diagram between a correlation coefficient and a static COP area corresponding to ascending the stairs.

FIG. 7A shows a relationship diagram of a correlation coefficient and a natural logarithm of a static COP corresponding to normal walking movements.

FIG. 7B shows a relationship diagram of a correlation coefficient and a natural logarithm of a static COP area corresponding to movements of ascending the stairs.

FIG. 8 shows a schematic diagram of thresholds of regression models.

FIG. 9 shows a flowchart of a method for monitoring postural and movement balance for fall prevention.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

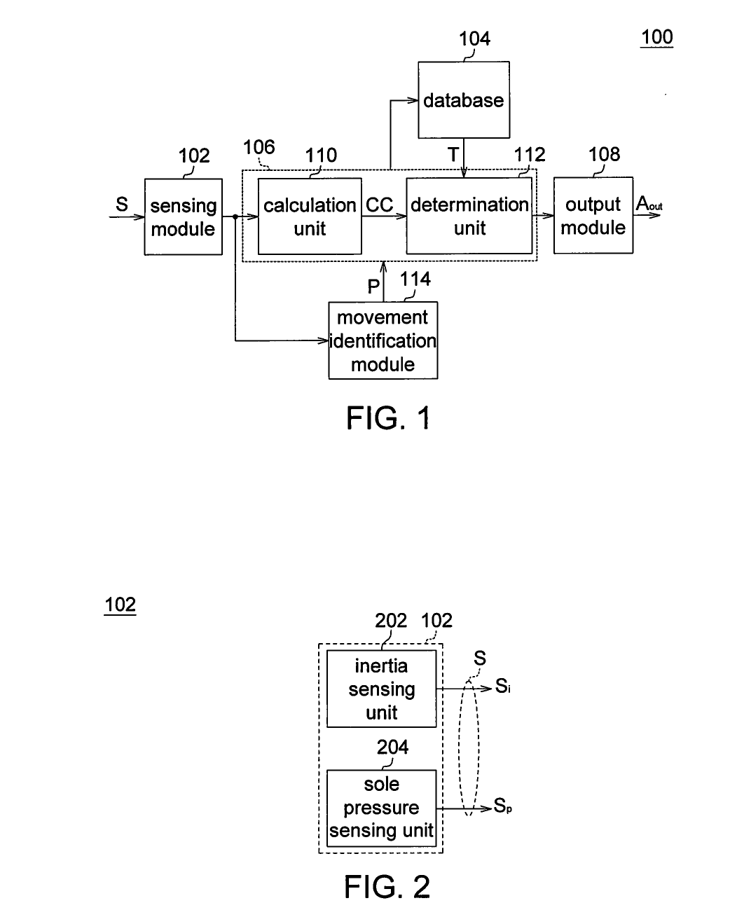

FIG. 1 shows a schematic diagram of a device 100 for monitoring postural and movement balance for fall prevention according to one embodiment. As shown in FIG. 1, the device 100 for monitoring postural and movement balance for fall prevention comprises a sensing module 102, a database 104, a calculation processing module 106 and an output module 108. For example, the sensing module 102 comprises a gyroscope, an accelerometer and a pressure sensor. For example, the database 104 is a hard drive, a memory card, or a device with a data storage capability. For example, the calculation processing module 106 is a central processing unit (CPU), or a device with an electronic computation capability. For example, the output module 108 is an alarm device, a device capable of outputting an alarm, or a circuit with a signal transmission capability for transmitting an alarm signal or information of the immediate balance states of the user self to a hospital, a monitoring center or related medical care staff.

The sensing module 102 obtains a plurality of sensing signals S of a human body. The database 104 stores at least one regression model. The calculation processing module 106 comprises a calculation unit 110 and a determination unit 112. The calculation unit 110 generates a center of mass (COM) signal and a center of pressure (COP) signal according to the sensing signals S, and calculates a correlation coefficient CC according to a mediolateral velocity of the COM signal and the COP signal. The determination unit 112 obtains a threshold T according to the regression model stored in the database 104, and determines whether the correlation coefficient CC is smaller than the threshold T. When the correlation coefficient CC is smaller than the threshold T, the calculation processing module 106 drives the output module 108 to output an alarm Aout. The alarm Aout may be presented in form of sound, light, or other means capable of generating an alert effect. Alternatively, the alarm Aout may be transmitted in form of a push message to related persons, e.g., family or medical care staff. Alternatively, the alarm Aout may be a driving signal for driving a device capable of maintaining human body balance. Further, in addition to outputting the alarm Aout by the output module 108 when the correlation coefficient CC is smaller than the threshold, other methods that determine whether to output the alarm Aout based on the comparison of the correlation coefficient CC and the threshold T are all encompassed within the scope of the disclosure.

In an embodiment, the device 100 for monitoring postural and movement balance further comprises a movement identification module 114. As shown in FIG. 1, the movement identification module 114 identifies a movement pattern P according to the sensing signals S. For example, the movement pattern P includes postures such as standing, stepping down, walking, ascending the stairs, descending the stairs, sitting down from standing, and standing up from sitting, for presenting a current movement of human body detected. From the database 104, the calculation processing module 106 then selects a regression model corresponding to the movement pattern P for calculation. In practice, the regression models corresponding to different movement patterns P may have corresponding thresholds T, respectively.

FIG. 2 shows a detailed block diagram of the sensing module 102 in FIG. 1. As shown in FIG. 2, the sensing module 102 comprises an inertia sensing unit 202 and a sole pressure sensing unit 204. The inertia sensing unit 202 obtains an inertia sensing signal Si. For example, the inertia sensing unit 202 may comprise a gyroscope and an accelerometer for measuring inertia sensing information corresponding to an angular velocity and an acceleration of a human movement. In an embodiment, the inertia sensing unit 202 may be disposed near center position of a COM of a human body, e.g., surface of the pelvis of a human body.

The sole pressure sensing unit 204 obtains a plurality of sole pressure signals Sp. For example, the sole pressure sensing unit 204 may comprise multiple pressure sensors, e.g., disposed on a shoe pad. Such that, when a user wears the shoe pad, the pressure sensors sense multiple sets of pressure information from a sole of the user and converts the same into a plurality of sole pressure signals Sp. In an embodiment, the pressure sensors are in a number of three or more.

The above inertia sensing signal Si and the sole pressure signals Sp, as regarded being included in the sensing signals S, are provided to the movement identification module 114 for subsequent processing to identify the movement pattern P of the human body, or provided to the calculation processing module 106 to model related kinematics of COM and COP of the human body as an inverted pendulum model. The correlation coefficient CC is determined further.

For example, the movement identification module 114 may perform a wavelet transform on the sensing signal Sp to identify the movement pattern P. In the so-called wavelet transform, a signal, through a scaling function and a wavelet function, is broken down into an approximated signal and a detail signal. The scaling function may be represented as

$${Φ}_{j , k} ( n ) = {2}^{- \frac{j}{2}} Φ ( {2}^{- j} n - k ) ,$$

and the wavelet function may be represented as

$${Ψ}_{j , k} ( n ) = {2}^{- \frac{j}{2}} Ψ ( {2}^{- j} n - k ) .$$

As such, a wavelet conversion is performed on a vertical acceleration a(t) of the inertia sensing signal Si for further characteristic value identification, which categorizes various movement patterns P.

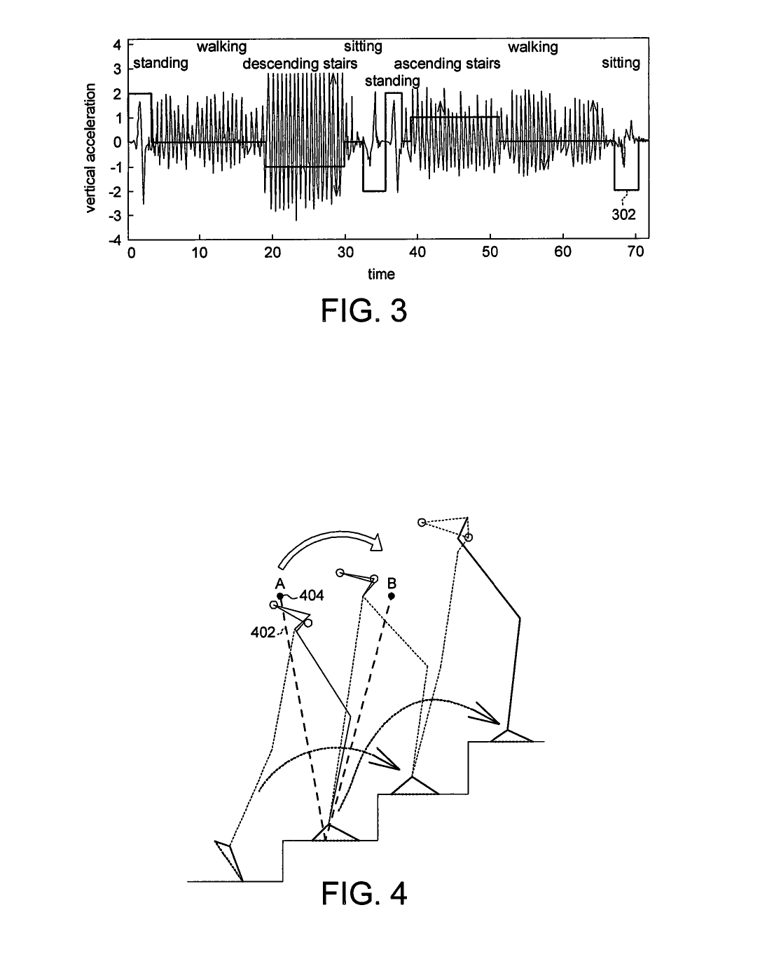

FIG. 3 shows a relationship diagram between the vertical acceleration a(t) and time. As seen from FIG. 3, the vertical acceleration a(t) is categorized into signal periods of standing, walking, ascending the stairs, descending the stairs and setting down according to the wavelet transform and the characteristic value identification (a curve 302).

After identifying the movement pattern P, the calculation processing module 106 performs an identification of a period of single limb support through the vertical acceleration a(t) of the inertia sensing signal Si, in order to subsequently model related kinematics of COM and COP of the human body by an inverted pendulum model, and to calculate the correlation coefficient CC of the mediolateral velocity of the COM signal and the COP signal.

FIG. 4 shows a schematic diagram of a period of single limb support when ascending the stairs by simulating a human body as an inverted pendulum model. As shown in FIG. 4, a virtual connecting rod 402 represents the inverted pendulum model of a human body. When an end point 404 of the virtual connecting rod 402 swings from a position A to a position B, a duration undergone may correspond to the period of single limb support when the human body ascends the stairs.

In an embodiment, an algorithm that the calculation processing module 106 identifies the period of single limb support is as follows.

A backward differentiation is performed on the vertical acceleration a(t) of the inertia sensing signal Si to obtain a function f(t). The function f(t) is organized into a step function a′(t) below:

$${a}^{′} ( t ) = \{ \begin{matrix}- 1 , f ( t ) < 0 \\ 0 , f ( t ) = 0 , f ( t ) = \frac{d a ( t )}{d t} \\ 1 , f ( t ) > 0\end{matrix}$$

Another backward differentiation is performed on the step function a′(t), which is then organized into another step function a″(t):

$${a}^{″} ( t ) = \{ \begin{matrix}\begin{matrix}1 , {f}^{′} ( t ) \neq 0 \\ 0 , {f}^{′} ( t ) = 0\end{matrix} & , {f}^{′} t = \frac{d {a}^{′} ( t )}{d t}\end{matrix}$$

The time point when the value of the step function a″(t) is zero and the time point when the vertical acceleration a(t) is greater than 1 are obtained, and a corresponding result is defined as a landing instant (T_{HS}). The time point when the value of the step function a″(t) is zero and the time point when the vertical acceleration is smaller than 1 are obtained, and a corresponding result is defined as a taking-off instant (T_{TO}). A signal period between the taking-off instant (T_{TO}) and the landing instant (T_{HS}) is the period of single limb support.

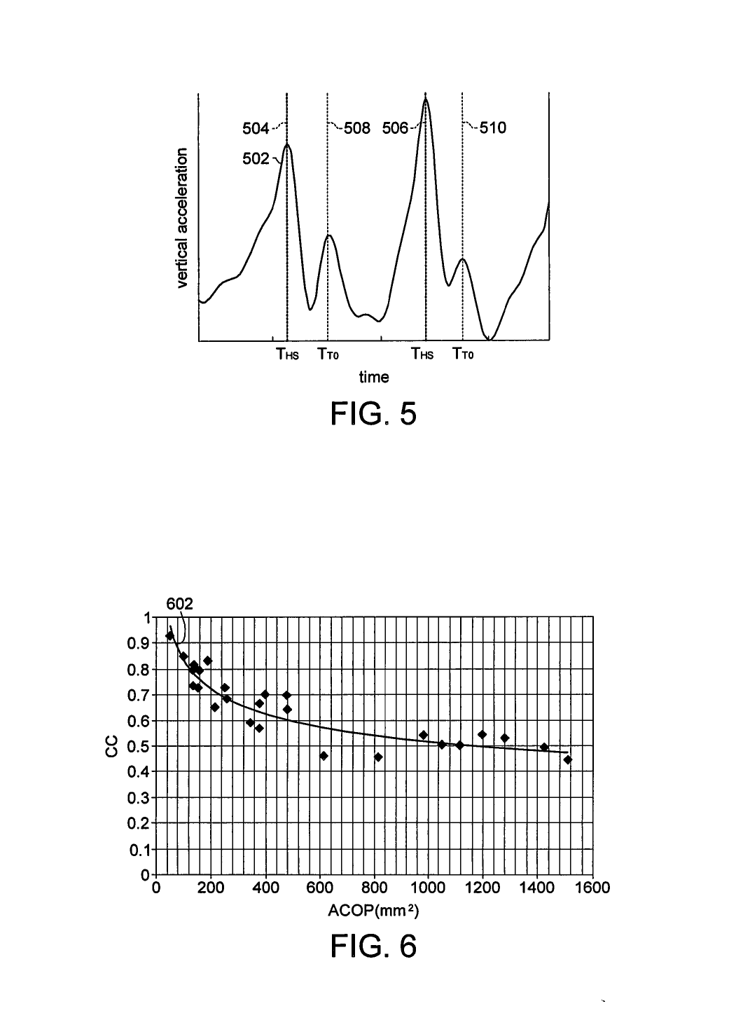

FIG. 5 shows a relationship diagram of the vertical acceleration a(t) of the inertia signal Si and time. As shown in FIG. 5, a curve 502 represents the vertical acceleration a(t) changing with time; time points corresponding to straight lines 504 and 506 represent the landing instant (T_{HS}); time points corresponding to straight lines 508 and 510 represent the taking-off instant (T_{TO}). A period from the time (T_{TO}) corresponding to the straight line 508 to the time (T_{HS}) corresponding to the straight line 506 is the period of single limb support.

Once the period of single limb support is determined, the related kinematics of COM and COP can be modeled as an inverted pendulum using the following transform algorithms.:

$${ρ}^{->} = {P}^{->} ( {T}_{HS} ) - {P}^{->} ( {T}_{TO} )$$

$${b}^{->} = \frac{[ {ρ}_{X} {ρ}_{Y} 0 ]}{\sqrt{{ρ}_{X}^{2} + {ρ}_{Y}^{2}}}$$

$$R = [ \begin{matrix}bx & 0 & by \\ by & 0 & - bx \\ 0 & 1 & 0\end{matrix} ]$$

$${{V}^{->}}_{\overline{COP}} = R \cdot \frac{d {P}^{->} ( t )}{d t} ( t = {T}_{HS} , {T}_{HS + 1} , … , {T}_{TO - 1} , {T}_{O} )$$

$${{V}^{->}}_{\overline{COM}} = R \cdot \int {a}^{->} ( t ) d t ( t = {T}_{HS} , {T}_{HS + 1} , … , {T}_{TO - 1} , {T}_{O} )$$

In the above equations, \vec{ρ} represents the direction vector of all the sole pressure signals Sp (represented by \vec{P}(T) in the above equations) of the period of single limb support from the beginning to the end. ρₓ and ρ_{y} represent the x-direction vector and the y-direction vector of the direction vector \vec{ρ} respectively. The z component (e.g., the component perpendicular to the ground) of the direction vector \vec{ρ} is then set as zero to obtain a unit vector \vec{b} parallel to the ground, where bₓ and by respectively represent the x-direction component and the y-direction component of the unit vector \vec{b}. The components of the unit vector \vec{b} are arranged into a rotation matrix R that describes a transformation relationship between a local coordinate system (walking coordinate system) and a global coordinate system (original coordinate system of the pressure insole) during the period of single limb support. The sole pressure signals Sp of the period of single limb support are differentiated and multiplied by the rotation matrix R to obtain a COP signal relative to a local coordinate system (represented by \vec{V} _{C̅O̅P̅} in the above equations). The vertical acceleration a(t) of the period of single limb support is integrated and multiplied by the rotation matrix R to obtain a COM signal relative to the local coordinate system (represented by \vec{V} _{C̅O̅P̅} in the above equations).

After the COM signal and the COP signal during movement are determined, the relative velocity of COM and COP may be further calculated under a local coordinate system. For example, x-axis and z-axis velocity under the local coordinate system represent the velocity of walking direction and mediolateral direction respectively.

According to the researches, the correlation coefficient CC of the mediolateral velocity of the COM signal and the COP signal is remarkably correlated to the movement balance during motion. That is, lower CC represents worse balance state during movement. Therefore, the correlation coefficient CC may be served as an index for determining a postural and movement balance of a human body.

FIG. 6 shows a relationship of the correlation coefficient CC and a static COP area (denoted as ACOP in the diagram) corresponding to a movement of ascending the stairs. It should be noted that, the static COP area determined from the equivalent ellipsoidal area of COP trajectories during static standings at different balance states, which represents the static balance of a human body. In other words, the larger COP area is determined the worse balance is shown of a human body (i.e., in an unbalanced state). In FIG. 6, the points that are discretely distributed represent distributed data of the correlation coefficient CC with respect to the static COP area. A curve 602 is a regression model established from fitting the static COP area with the distribution of the correlation coefficient CC. The regression model displays a decreasing index function CC=−0.071 ln(ACOP)+0.998, and a determination coefficient (R²) of regression analysis is 0.83. As observed from the curve 602, the correlation coefficient CC of the mediolateral velocity of the COM signal and the COP signal gets lower under an increasingly unbalanced state (as the static COP area gets larger).

In an embodiment, the relationship between the correlation coefficient CC and the static COP area of amount of subjects is first obtained to establish one or multiple regression models in the database 104. For example, the subjects may first carry out a laboratorial postural balance experiment. In the experiment, bodies of the subjects are attached with multiple (e.g., 39) reflective balls, with the subjects standing still on a force plate to measure the COP trajectory to determine the equivalent area. The subjects are then required to step over the force plate with a normal walking velocity to measure the correlation coefficient CC of the mediolateral velocity of the COM signal and the COP signal. As such, the distribution data of multiple correlation coefficients CC at different balance state with respect to the static COP areas can be obtained using above measurement process. The distribution data are computed by regression to establish regression models corresponding to normal walking movements of the subjects. In addition to the above embodiment, other methods may also be adopted to establish regression models of other movement patterns P. Associated details are similar to the above embodiment, and shall be omitted herein. Further, given that the distribution data corresponding to different movement patterns P are computed by regression algorithms, one regression model may correspond to two or more movement patterns P.

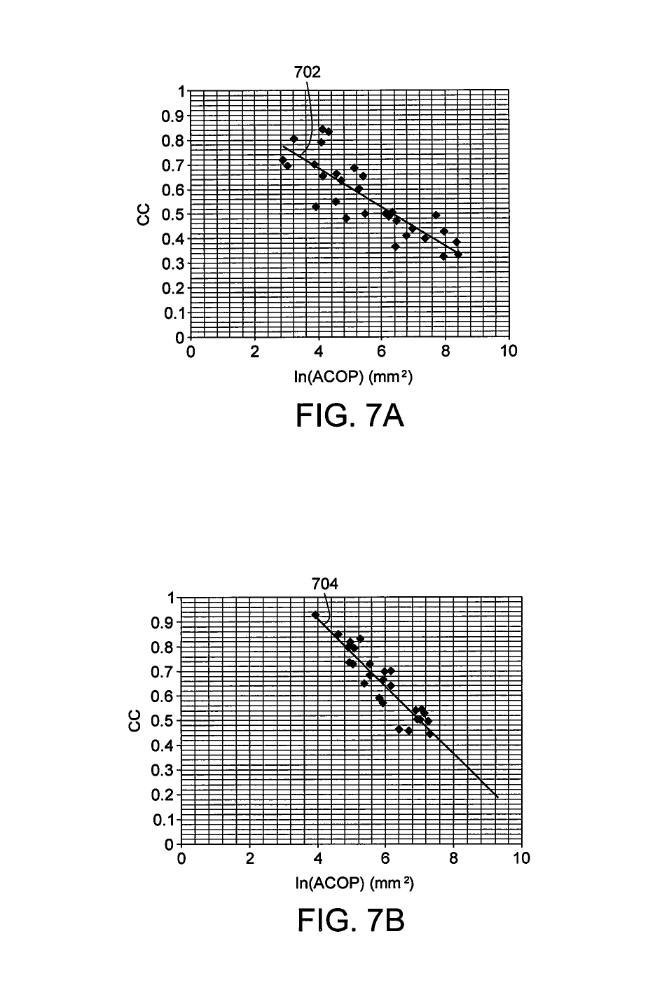

In an alternative embodiment, the regression model may represent the relationship between the correlation coefficient CC and a natural logarithm of the static COP area to obtain a linear prediction model. Take FIGS. 7A and 7B depicting relationship diagrams between the correlation coefficient CC and natural logarithms (indicated by ln(ACOP) in the diagrams) of the static COP area for example. In FIG. 7A, a straight line 702 represents a regression model in a function CC=0.0785*ln(ACOP)+0.9979, and the determination coefficient (R²) is 0.7148. In FIG. 7B, a straight line 704 represents a regression model in a function CC=0.1363 ln(ACOP)+1.457, and the determination coefficient (R²) is 0.8558. It is displayed that, the distribution data is highly correlated in a linear manner.

The linear regression model may also be categorized according to different subject groups. For example, the regression model may satisfy the following equation:

ln(ACOP)=1.65−6.06*ln(CC)+0.5*G1+0.88*G2+0.9*G3

In the equation above, for example, coefficients G1, G2 and G3 are as in the table below:

| Group | G1 | G2 | G3 |
|---|---|---|---|
| Youth | 0 | 0 | 0 |
| Middle-aged | 1 | 0 | 0 |
| Elderly | 0 | 1 | 0 |
| Elderly that have | 0 | 0 | 1 |
| fallen within past one year | | | |

As such, subjects of different age groups respectively correspond to one linear regression model. Through the linear regression model, the corresponding balance state (the static COP area) may be calculated by the dynamic correlation coefficient CC during movement.

Having established the regression model, the determination unit 112 may obtain the threshold T according to the regression model, and determine whether the correlation coefficient CC is smaller than the threshold T. Under normal circumstances, the chance of a human body in an unbalance state of having fallen/about to fall is small, and so the threshold T may be designed in a way that, 5% (or less) of the distribution data falls in a region where the correlation coefficient CC is smaller than the threshold T. Thus, when the determination unit 112 determines that the correlation coefficient CC is smaller than the threshold, it is regarded that a person wearing the device (wearer) is in an unbalanced state of having fall/about to fall.

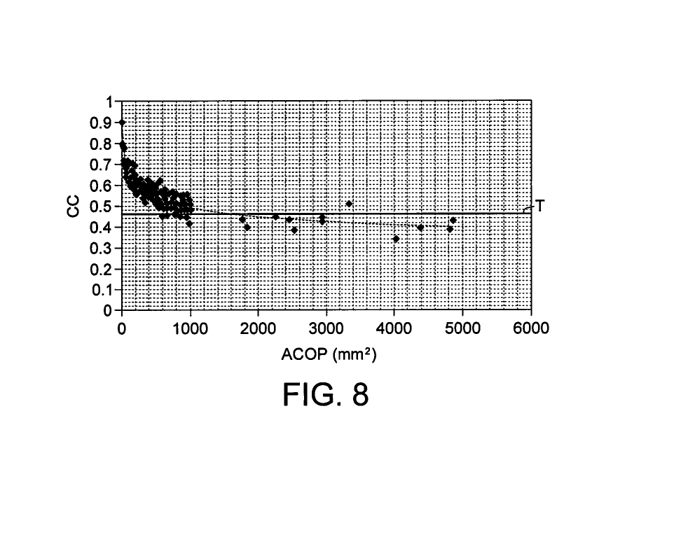

FIG. 8 shows a schematic diagram of the threshold T of a regression model. As seen from FIG. 8, the threshold T is set to 0.45. In such regression model, majority of the distribution data falls within regions where the correlation coefficient CC is greater than the threshold T, with the remaining minority of the distribution data being located within regions where the correlation coefficient CC is smaller than the threshold T. It should be noted that, instead of setting the threshold T to 0.45, the threshold T may be adjusted into different values according to different requirements or different groups of wearers.

In one embodiment, the threshold T may be designed according to the static balance of a human body. That is to say, by designing various different static balance test conditions and obtaining differences of natural logarithms (ln(ACOP) of the static COP area under these environments, the threshold T may be determined. For example, the static balance test include four conditions of standing with eyes open (A), standing with eyes shut (B), standing after turning five rounds on an original standing spot (C), and standing after turning ten rounds on an original standing spot (D). The natural logarithms of the corresponding static COP area of normal young people under such test conditions are measured for reference of determining the threshold T. For example, the measured results are as in the table below:

| Test conditions | In(ACOP) |
|---|---|
| Standing with eyes open (A) | <5 |
| Standing with eyes shut (B) | 5~6 |
| Standing after turning five rounds on | 6~7 |
| original standing spot (C) | |
| Standing after turning ten rounds on | >7 |
| original standing spot (D) | |

At this point, assuming that the natural logarithm of the static COP area is 6.5, it means that the corresponding standing balance capability is between the conditions of standing with eyes shut (B) and standing after turning five rounds on an original standing spot (C). In one embodiment, the threshold T may be designed as 6.5 (mm²). The determination unit 112 determines whether the natural logarithm of the static COP area of the wearer is greater than the threshold T, and the calculation processing module 106 drives the output module 108 to output the alarm Aout if so.

In one embodiment, the device 100 for monitoring postural and movement balance for fall prevention has a personalized capability for dynamically updating the database 104. That is to say, the calculation processing module 106 is capable of calculating the current static COP area corresponding to a standing posture of a wearer, and combining the measured correlation coefficient CC to update and correct the regression model originally stored in the database 104. As such, the updated regression model may better match the actual balance state of the wearer.

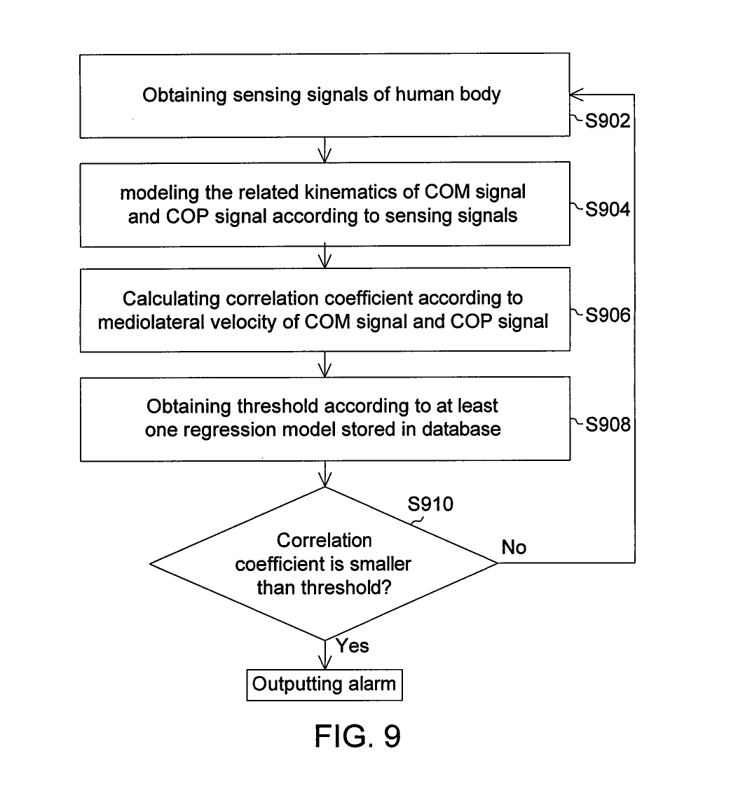

A method for monitoring postural and movement balance for fall prevention is further provided according to an embodiment. The method is applicable to the device 100 for monitoring postural and movement balance for fall prevention. FIG. 9 shows a flowchart of a method for monitoring postural and movement balance for fall prevention. The method comprises steps S902, S904, S906, S908 and S910. In step S902, a plurality of sensing signals S of a human body are obtained. In step S904, a COM signal and a COP signal are generated according to the sensing signals S. In step S906, a correlation coefficient CC is calculated according to a mediolateral velocity of the COM signal and COP signal. In step S908, a threshold T is obtained according to at least one regression model stored in a database 104. In step S910, whether the correlation coefficient CC is smaller than the threshold T is determined. An alarm Aout is output when the correlation coefficient CC is smaller than the threshold T, or else step S902 is iterated.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for monitoring postural and movement balance for fall prevention, comprising:
obtaining a plurality of sensing signals of a human body;
modeling related kinematics of a center of mass (COM) signal and a center of pressure (COP) signal according to the sensing signals;
calculating a correlation coefficient according to a mediolateral velocity of the COM signal and the COP signal;
obtaining a threshold according to at least one regression model stored in a database;
determining whether the correlation coefficient is smaller than the threshold;
outputting an alarm when the correlation coefficient is smaller than the threshold.

2. The method according to claim 1, wherein the sensing signals comprise an inertia sensing signal and a plurality of sole pressure signals.

3. The method according to claim 2, further comprising:
identifying a movement pattern according to the inertia signal;
in the step of obtaining the threshold, selecting the regression model corresponding to the movement pattern from the database according to the movement pattern.

4. The method according to claim 3, wherein the step of identifying the movement pattern comprises:
performing a wavelet transformation on the inertia signal to identify the movement pattern.

5. The method according to claim 4, wherein the movement pattern comprises standing, stepping down, walking, ascending stairs, descending stairs, standing up from sitting, sitting down from standing, and running.

6. The method according to claim 2, wherein the step of modeling related kinematics of the COM signal and the COP signal is performed through calculation by use of an inverted pendulum model.

7. The method according to claim 6, further comprising:
determining a period of single limb support for modeling the inverted pendulum model according to a vertical acceleration of the inertia signal.

8. The method according to claim 1, wherein the at least one regression model represents a relationship between the correlation coefficient in relation to different balance states and COP areas measured during static standings respectively, wherein the COP areas are determined from equivalent areas of COP trajectories.

9. The method according to claim 8, further comprising:
during a static posture, calculating the correlation coefficient and a corresponding COP area according to the sensing signals, and correcting the at least one regression model according to the correlation coefficient and the corresponding COP area.

10. A device for monitoring postural and movement balance for fall prevention, comprising:
a sensing module, for obtaining a plurality of sensing signals of a human body;
a database, for storing at least one regression model; and
a calculation processing module, comprising:
a calculation unit, for modeling related kinematics of a COM signal and a COP signal according to the sensing signals, and calculating a correlation coefficient according to a mediolateral velocity of the COM signal and the COP signal;
a determination unit, for obtaining a threshold according to at least one regression model stored in a database, and determining whether the correlation coefficient is smaller than the threshold;
an output module, for outputting an alarm when the correlation coefficient is smaller than the threshold.

11. The device according to claim 10, wherein the sensing module comprises:
an inertia sensing unit, for obtaining an inertia sensing signal; and
a sole pressure sensing unit, for obtaining a plurality of sole sensing signals.

12. The device according to claim 11, wherein the inertia sensing unit comprises a gyroscope and an accelerometer.

13. The device according to claim 11, wherein the inertia sensing unit is attached near the position of COM on the human body.

14. The device according to claim 11, wherein the sole pressure sensing unit comprises a plurality of pressure sensors disposed on a shoe pad.

15. The device according to claim 14, wherein the pressure sensors are in a number of at least three.

16. The device according to claim 11, further comprising:
a movement identification module, for identifying a movement pattern according to the inertia sensing signal;
wherein, the calculation processing module selects the regression model corresponding to the movement pattern from the database according to the movement pattern.

17. The device according to claim 16, wherein the inertia sensing signal performs a wavelet transformation on the inertia signal to identify the movement pattern.

18. The device according to claim 17, wherein the movement pattern comprises standing, stepping down, walking, ascending stairs, descending stairs, standing up from sitting, sitting down from standing, and running.

19. The device according to claim 11, wherein the calculation processing module models the COM signal and the COP signal according to an inverted pendulum model.

20. The device according to claim 19, wherein the calculation processing module determines a period of single limb support for the inverted pendulum model according to a vertical acceleration of the inertia sensing signal.

21. The device according to claim 10, wherein the at least one regression model represents a relationship between the correlation coefficient in relation to different balance states and COP areas measured during static standings respectively, wherein the COP areas are determined from equivalent areas of COP trajectories.

22. The device according to claim 21, wherein the calculation processing module calculates the correlation coefficient and a corresponding COP area according to the sensing signals during a static posture, and corrects the at least regression model stored in the database according to the corresponding COP area.
